(19) United States
(12) Patent Application Publication
(10) Pub. No.: US 2015/0025407 A1
(43) Pub. Date: Jan. 22, 2015

(54) DEVICES AND METHODS FOR GENERATING AN ARTIFICIAL EXHALATION PROFILE

(71) Applicant: Aerocrine AB, Solna (SE)

(72) Inventors: Rudiger Eichler, Zellingen (DE); Kjell Alving, Uppsala (SE)

(21) Appl. No.: 14/509,521

(22) Filed: Oct. 8, 2014

Related U.S. Application Data
(63) Continuation of application No. 14/236,626.
(60) Provisional application No. 61/526,427, filed on Aug. 23, 2011.

(30) Foreign Application Priority Data
Aug. 23, 2011 (SE) 1150761-3

(51) Int. Cl. A61B 5/097; A62B 23/06; A61M 16/08; G01N 33/00; A61M 16/00; A61B 5/08

(52) U.S. Cl. CPC A61B 5/097; A61M 16/0069; A61M 16/0003; A61B 5/082; A61M 16/085; G01N 33/0037; A62B 23/06; A61M 2016/0042; A61M 2016/0027; A61M 2205/3344; A61M 2205/3334; A61M 2205/3337; A61M 2240/00; A61M 2210/0625; A61M 2210/0618
USPC 600/532; 600/543; 128/204.23

(57) ABSTRACT

Methods and devices for creating an artificial exhalation profile, for example for use in the sampling of exhaled breath or air from the nasal cavity from a mammal, wherein said mammal exhales into a device comprising a flow channel, a pressure sensor, a flow sensor, a control unit, and means for creating an exhalation flow, wherein said means for creating an exhalation flow maintain an exhalation flow at one or more pre-determined flow rate (rates), within a predetermined interval, substantially independent of exhalation pressure.

### RELATED APPLICATION DATA

This application is a continuation of U.S. patent application Ser. No. 14/236,626, filed Feb. 1, 2014, which is a National Stage entry application of International Application No. PCT/EP2012/066425, filed Aug. 23, 2012, which claims the benefit of U.S. Provisional Patent Application No. 61/526,427, filed Aug. 23, 2011 and Swedish Application No. 1150761-3, filed Aug. 23, 2011, the disclosures of which are incorporated herein by reference in their entireties.

### TECHNICAL FIELD

The present invention relates to the field of breath analysis, and in particular to devices and methods for controlling the flow rate when taking a sample from exhaled breath and/or air from the nasal cavity of mammals. Techniques for sampling and analyzing exhaled breath and/or air from the nasal cavity, and in particular components therein, have high relevance in clinical applications such as the diagnosis and monitoring of pathologies, as well as in research, preventive health care and exercise.

### BACKGROUND

A variety of components can be detected in exhaled breath, and/or in air from the nasal cavity, ranging from gaseous components such as carbon monoxide and nitric oxide, to mention two non-limiting examples; to particulate matter such as for example cells, microbes, and macromolecules; and volatile organic and inorganic compounds. Whereas the gaseous components can be detected in the gas phase, other components may require additional steps, such as the collection of breath condensate, and/or other collection techniques, such as filtering.

There is ongoing research regarding exhaled breath condensate, as the collection of exhaled breath condensate is recognized as a noninvasive method to obtaining samples from the airways and the lungs. Exhaled breath condensate has been found to contain large number of mediators including adenosine, ammonia, hydrogen peroxide, isoprostanes, interleukins, leukotrienes, nitrogen oxides, peptides and cytokines. The concentrations of these mediators are influenced by lung diseases and modulated by therapeutic interventions (Horwath et al., Exhaled breath condensate: methodological recommendations and unresolved questions, in Eur Respir J, 2005, September; 26(3):523-48). A device and method for collecting breath condensate has been disclosed in WO 2011/038726.

The detection of various components in exhaled breath and the scientific work associating these with different pathologies has led to a rapid growth in the field of exhaled breath analysis. Currently, the main focus is on the measurement of nitric oxide, known to be a diagnostic marker of inflammation (see e.g. WO 93/05709; WO 95/02181).

In order to guarantee accuracy and repeatability, and almost independently of the component to be determined, it is desirable to control the exhalation parameters such as flow, volume, pressure and duration of exhalation, in order to minimize or preferably even eliminate variations in these parameters. In the alternative, it is necessary to record these parameters in order to take them into account when calculating the result.

In 1997 the European Respiratory Journal published guidelines (ERS Task Force Report 10: 1683-1693) for the standardization of NO measurements in order to allow their rapid introduction into clinical practice. Later, in 1999, the American Thoracic Society (ATS) published guidelines for clinical NO measurements. These have since been updated and supplemented (An Official ATS Clinical Practice Guideline: Interpretation of Exhaled Nitric Oxide Levels (FeNO) for Clinical Applications, Am. J. Respir. Crit. Care Med. 2011 184: 602-615).

An apparatus for diagnostic gas analysis which supports compliance with the above guidelines is disclosed in WO 2004/023997, wherein the subject exhales into the apparatus at a predetermined flow rate and pressure, and said apparatus comprises means for temporarily storing a portion of the exhaled air and means for feeding said stored portion to a sensor for determining the concentration of nitric oxide, wherein the sample is fed to the sensor during a period of time longer than the duration of the exhalation and at a flow rate below the exhalation flow rate.

Another method and device for improved sampling and measurement of exhaled nitric oxide originating from different parts of the lungs is disclosed in WO 2002/091921. Air exhaled by a subject is received in a tube of the measuring device, wherein the exhalation flow rate within the tube is measured to adjust tube flow resistance in accordance with the measured exhalation flow rate in the tube in order to keep the flow rate on a prescribed level.

Further, WO 2008/106961 discloses another apparatus for determining components of exhaled breath, where a positive end-expiratory pressure valve (PEEP valve) is used to maintain the exhalation pressure and flow within desired intervals.

WO 2006/086323 discloses an apparatus determining a component in exhaled breath, where the flow rate of a gaseous sample of exhaled breath through an analytical device is controlled by a pump, and in certain embodiments, two pumps. Placement of the analyte sensor in a secondary stream branching off of the primary stream through the device offers further control over the manner, duration, and quantity of the breath that is placed in contact with the sensor.

Regardless of their advantages, the above apparatuses require the conscious cooperation of the subject to be sampled. Children, elderly and sick may have difficulties complying with the requirements for performing the test, e.g. the ATS guidelines. For infants, small children, very weak, physically or mentally incapacitated or even unconscious subjects, compliance is of course not to be expected. In fact, it is held that it is extremely difficult to obtain valid fractional exhaled nitric oxide (FeNO) measurements during tidal breathing in preschool children (between 2 and 5 years of age) without sedation even when visual cues and animation is used to motivate these small children (Barroso N. C. et al., Exhaled Nitric Oxide in Children: A Noninvasive Marker of Airway Inflammation, Arch Bronconeumol. 2008; 44(1): 41-51).

In another study, investigating the concentration of nitric oxide in exhaled breath of infants, aged 3 to 24 months, forced expiration was achieved by compressing the chest and abdomen with an inflatable jacket to transmit to the airway a pressure of 20 cm H₂O above inflation pressure at end-inspiration (Wildhaber J. H. et al., Measurements of Exhaled Nitric Oxide with the Single-Breath Technique and Positive Expiratory Pressure in Infants, Am J Respir Crit Care Med Vol 159. pp 74-78, 1999).

U.S. Pat. No. 6,067,983 discloses an apparatus and method for controlled flow sampling from the airway including a mouthpiece, or a connector attached to a tube inserted in the subject's trachea, either of which is used to capture gases from the subject's airway. Attached to the mouthpiece or the connector is a total airway occlusion. A pump or vacuum source, maintained at a lower pressure than the pressure inside the airway, is connected to the total airway occlusion, pulling gas out of the airway independent of the subject's volition. The flow is maintained at a substantially constant rate chosen by the operator through control over the source of low pressure. As gases flow out of the airway, they flow through a gas analyzer which measures desired properties of the gas.

Positive airway pressure (PAP) ventilation and continuous positive airway pressure (CPAP) ventilation was initially developed for the treatment of obstructive sleep apnea, but has later found widespread use also in the clinical setting, as a form of ventilation, improving the gas exchange and reducing the breathing effort for the subject by assisting during inhalation.

One application of the PAP technique is the so called variable or bi-level PAP (BIPAP) ventilation, where the inspiratory PAP (IPAP) is set at a higher level, and the expiratory PAP (EPAP) is set at a lower level, for easier and more comfortable exhalation by preventing airway closure. Further, PAP ventilation is frequently performed in a so called “spontaneous” mode, meaning that the device triggers IPAP when flow sensors detect a spontaneous inspiratory effort.

JP Kokai 1998-048206 (application no. 1996-216653) discloses a breath sampling and analyzing device comprising a breath sensor and a breath introduction mechanism, including a suction pump, controlled by said breath sensor. The pump is activated only when the breath sensor detects the flow of exhaled breath, a pressure increase, or an increase in temperature. The purpose of this set-up is to guarantee that a sample of exhaled breath is taken only when a subject performs a proper exhalation into the device. Applied to breath analysis in drug enforcement or drunk driving, this protects against various forms of deception. A closer study of this document, including FIG. 1, however reveals that said pump only transports the sample from a breath sampling tube to an analysis unit. The breath sampling tube is open, and has no means to control the flow of exhalation.

### SUMMARY

One objective is to improve and simplify the sampling and analysis of exhaled breath, and/or air from the nasal cavity, while maintaining comfort, compliance and repeatability at a high level.

Another objective, linked to the above, is to make it possible to control the exhalation flow to defined values, suitable for different measurements. In the case of nasal sampling, instead of exhalation flow, the term aspiration flow or only flow will be used.

Yet another objective, also liked to the above objects, is to make available a method and device for use in the sampling of exhaled breath, and/or air from the nasal cavity, from a mammal, where the method and device can be applied to any subject, regardless of age and capability to comply with instructions, such as infants, small children, sick and elderly, or even non-human animals.

Another objective, liked to the above objects, is to offer clear and easily understandable incentives and/or instructions for starting and maintaining exhalation, in a manner that supports compliance, accuracy and repeatability.

These objectives and others are met by a method and device or system according to the attached claims, incorporated herein by reference.

According to one embodiment, the invention makes available a method or at least a method step in the sampling of exhaled breath from a mammal, wherein said mammal exhales into a system comprising a flow channel, a pressure sensor, a flow sensor, a control unit, and means for creating an exhalation flow, such as a pump or a fan, wherein the pressure sensor measures the mouth pressure generated by the mammal and the flow sensor measures exhalation flow in said flow channel.

The control unit receives signals from the pressure sensor and the flow sensor, and when the measured mouth pressure exceeds a predetermined value, or when said pressure sensor or said flow sensor detects a pre-determined increase in pressure or flow, the control unit sends a signal to activate said pump or fan for creating an exhalation flow.

Once activated, said pump or fan for creating an exhalation flow, such as a pump or a fan, maintain a targeted exhalation flow of at least one pre-determined flow value, substantially independent of exhalation pressure.

Said pump or fan is then deactivated, and the flow stopped, when a predetermined time has lapsed, or when a predetermined exhalation volume has been exhaled, or when the pressure sensor detects a pre-determined decrease in mouth pressure.

In the above embodiment, preferably the pump or fan is activated when said pressure sensor detects a pre-determined increase in mouth pressure.

The exhalation flow rate is maintained at least at one value, or allowed to vary, within the interval of about 1 to about 1000 ml/s, depending on the specific application of the method. Preferably the exhalation flow rate is maintained at least at one value within said interval of about 1 to about 1000 ml/s, or about 5 to about 600 ml/s, with an accuracy of at least +/−10% of the desired value. Other preferred intervals are for example 1-100 ml/s, 20-350 ml/s, 40-400 ml/s, 40-600 ml/s and different sub-intervals thereof. The flow can for example be kept sequentially at 50, 100 and 150 ml/s for predetermined periods of time. Most preferably the flow is maintained at a desired level with an accuracy of at least +/−10%, preferably at least +/−5% of the desired value. Thus, the term “maintained” in this context also encompasses embodiments where the flow is allowed to vary within the indicated intervals, and with the indicated accuracy.

In the above embodiment, the mouth pressure is maintained at at least about 3 mbar, preferably in the interval of about 3 to about 40 mbar, more preferably 7-20 mbar, in order to make sure that the velum is closed, isolating the nasal airways from the oral cavity and excluding contamination of orally exhaled air with air originating from the nasal cavity. The lower limit of the interval is critical, in order to make sure that the velum is closed. The upper limit of the pressure interval is less critical, and dependent of the construction of the device. It is possible to construct the device which tolerates high pressures, within the limits that can be expected. A male adult can produce a maximal expiratory pressure of about 150 mbar, but in a setting where instructions or feed-back is given, so high pressures are not expected.

Further, in the above embodiment, and freely combinable with the other embodiments presented herein, said flow is maintained at two or more sequential and pre-determined flow values, each for a predetermined period of time or volume. The flow can for example be kept sequentially at 50, 100 and 150 ml/s for predetermined periods of time.

Preferably the method also involves the presentation of feed-back, constituting an incentive for the mammal to perform the required inhalation and exhalation maneuvers. The incentive in particular encourages and motivates the mammal to initiate the exhalation and to maintain at least a required minimum pressure or flow for the time needed for the sampling. Examples of feed-back and incentives include audio signals, visual signals, tangible signals and combinations thereof.

One example of a visual feed-back is the traffic light set-up, where different colors can be used to indicate how well the exhalation is performed. The traffic light set up can also be used to indicate the start and stop of the required exhalation. In addition to different colors, or as an alternative thereto, the intensity and duration of the color signal can be varied. A weak or slowly flickering light can be used as an indication that the performance is sub-optimal, whereas a strong, steady light can be used as a confirmation of a proper performance. A rapidly flickering light can be used to indicate that a desired level (e.g. pressure, flow) has been exceeded. Green and red light can be used to signal start and stop and so on. Preferably the visual incentive is presented in the form of an animation, showing for example a thermometer or speedometer balancing between a low range, a “proper” range, and a high range.

Other examples of animations include tasks to be completed, a balloon to be inflated, an object to be blown across a certain distance indicated on a display or balanced in the middle of an interval indicated on a display (for example a feather, down, leaf, soap bubble, butterfly, dandelion seed, cloud etc). The animation of easily understandable tasks to be completed surprisingly functions both as a strong incentive, and as a good approach to discourage deviations from the desired performance. A good example is the inflation of a balloon, where a shriveling balloon clearly shows that the exhalation is too week, and a bursting balloon (for example accompanied by an acoustic signal: a bang!) can indicate the successful completion of the sampling and that the exhalation can be stopped. Similarly, it is easily understood that soap bubble should not be allowed to be resting on the ground (e.g. the lower limit of the display), nor should it be pressed up against the ceiling (the upper limit of the display).

An audio signal can be varied in a similar fashion, and by changing loudness and frequency, information about performance can be conveyed. For example an intermittent, low-pitched sound can be used as encouragement to exhale harder, whereas an intermittent, high-pitched sound can be used to indicate that a desired level (e.g. pressure, flow) has been exceeded. Alternatively, two or more audio signals are used, for example a hissing sound when a balloon is being inflated, and a loud bang when it bursts.

It is also possible to make the device or parts thereof vibrate, and again, a low-frequency vibration can be used to indicate sub-optimal performance, and a high-frequency vibration can be used to indicate that a certain threshold value has been exceeded.

Preferably visual signals, audio signals, and other signals, e.g. vibration, are combined and incorporated into an animation. The technology is readily available and already in use for example in game consoles, such as flight simulators, driving simulators etc.

The feed-back can also be realized analogously, or mechanically. It is for example conceived that a whistle is tuned so, that it gives a low, buzzing tone at low flow rates, a clear, even tone at the desired flow rate, and a high, shrill tone when the desired flow rate is exceeded.

The above methods for giving feed-back can be used with cooperative patients, i.e. from the age of about 3 years and upwards. Cooperative in this context means that the person is capable of understanding and following simple instructions. It is conceived that the feed-back can be given in such as fashion that cooperation and correct performance will be intuitive.

According to another embodiment, the invention makes available a method for creating an artificial exhalation profile, wherein said mammal exhales into a system comprising a flow channel, a pressure sensor, a flow sensor, a control unit, and a pump or fan for creating a flow, wherein the pressure sensor measures the mouth pressure generated by the mammal and the flow sensor measures the flow of exhalation air in said flow channel.

The control unit receives signals from the pressure sensor and the flow sensor, and when the measured mouth pressure exceeds a predetermined value, or when said pressure sensor or said flow sensor detects a pre-determined increase in pressure or flow, the control unit sends a signal to activate said pump or fan for creating an exhalation flow.

Once activated, said pump or fan for creating an exhalation flow, such as a pump or a fan, maintain a targeted exhalation flow of at least one pre-determined flow value, substantially independent of exhalation pressure.

Said pump or fan is then deactivated, and the flow stopped, when a predetermined time has lapsed, or when a predetermined exhalation volume has been exhaled, or when the pressure sensor detects a pre-determined decrease in mouth pressure.

In the above embodiment, preferably the pump or fan is activated when said pressure sensor detects a pre-determined increase in mouth pressure.

The exhalation flow rate is maintained at least at one value, or allowed to vary, within the interval of about 1 to about 1000 ml/s, depending on the specific application of the method. Preferably the exhalation flow rate is maintained at least at one value within said interval of about 1 to about 1000 ml/s, or about 5 to about 600 ml/s, with an accuracy of at least +/−10% of the desired value. Other preferred intervals are for example 1-100 ml/s, 20-350 ml/s, 40-400 ml/s, 40-600 ml/s and different sub-intervals thereof. The flow can for example be kept sequentially at 50, 100 and 150 ml/s for predetermined periods of time. Preferably the flow is maintained at, or allowed to vary around, a desired level with an accuracy of at least +/−10%, more preferably at least +/−5% of the desired value.

In the above embodiment, similarly as in the previous embodiment, and freely combinable with the other embodiments, the mouth pressure is maintained above about 3 mbar, preferably within an interval of about 3 to about 40 mbar, more preferably 7-20 mbar, in order to make sure that the velum is closed, isolating the nasal airways from the oral cavity and excluding contamination of orally exhaled air with air originating from the nasal cavity.

In the above embodiment, said flow is maintained at two or more sequential and pre-determined flow values, each for a predetermined period of time or volume.

The method according to any one of the embodiments disclosed herein can be applied to any mammal, but preferably said mammal is a human. Preferably, and applicable to any one of the embodiments disclosed herein, the human subject is chosen from the group including infants, small children, elderly, demented, mentally or physically disabled, and healthy.

Preferably also this embodiment involves the presentation of feed-back, constituting an incentive for the mammal to perform the required inhalation and exhalation maneuvers. The principles and methods for giving feed-back are as described above, in relation to the different embodiments.

According to another embodiment, the invention makes available a device or system for creating an artificial exhalation profile, comprising an inlet for receiving exhaled air from said mammal, a flow channel, a pressure sensor, a flow sensor, a control unit, and a pump or fan for creating an exhalation flow, wherein said pressure sensor and/or flow sensor are adapted to supply signals to the control unit, and the control unit controls the pump or fan in such manner, that said pump or fan is activated when said pressure and/or flow sensor detects a predetermined increase in mouth pressure and/or flow, and deactivated when said pressure sensor detects a predetermined decrease in mouth pressure.

According to yet another embodiment, the invention makes available a device or system for taking a sample of exhaled air from a mammal, comprising an inlet for receiving exhaled air from said mammal, a flow channel, a pressure sensor, a flow sensor, a control unit, and a pump or fan for creating an exhalation flow, wherein said pressure sensor and/or flow sensor are adapted to supply signals to the control unit, and the control unit controls the pump or fan in such manner, that said pump or fan is activated when said pressure and/or flow sensor detects a predetermined increase in mouth pressure and/or flow, and deactivated when said pressure sensor detects a predetermined decrease in mouth pressure.

In the above embodiments, said pump or fan for creating an exhalation flow is preferably connected to said pressure sensor in such manner, that said pump or fan is activated when said pressure sensor detects a predetermined increase in mouth pressure generated by the mammal.

In the above embodiments and others disclosed herein, said pump or fan is adapted for maintaining a predetermined flow within the interval of about 1 to about 1000 ml/s, or about 5 to about 600 ml/s, with an accuracy of at least +/−10% of the desired value. Other preferred intervals are for example 1-100 ml/s, 20-350 ml/s, 40-400 ml/s, 40-600 ml/s and different sub-intervals thereof.

Suitable pumps include positive displacement pumps or blowers, such as diaphragm pumps, plunger pumps, and gear pumps. Preferably said pump is a diaphragm pump, also called membrane pump. The advantage of a positive displacement pump is that it cannot be overridden by the subject.

In a preferred embodiment, freely combinable with the other embodiments, said predetermined flow is flexible, in the sense that multiple flow rates within the interval of about 1 to about 1000 ml/s can be maintained for predetermined periods of time. Preferably the flow is maintained at least at one predetermined flow with an accuracy of +/−10%. More preferably each flow is maintained with an accuracy of at least +/−5% of the desired value.

According to yet another embodiment, the invention makes available a device or system for determining the concentration of a component in exhaled air from a mammal, comprising an inlet for receiving exhaled air from said mammal, a flow channel, a pressure sensor, a flow sensor, a control unit, and a pump or fan for creating an exhalation flow and a sensor specific for the component to be determined, wherein said pressure sensor and/or flow sensor are adapted to supply signals to the control unit, and the control unit controls the pump or fan in such manner, that said pump or fan is activated when said pressure and/or flow sensor detects a predetermined increase in mouth pressure and/or flow, and deactivated when said pressure sensor detects a predetermined decrease in mouth pressure, and wherein said flow of exhaled air is brought in contact with said sensor specific for the component to be determined.

In the above embodiment, said pump or fan for creating a flow is connected to said pressure sensor in such manner that said pump or fan is activated when said pressure sensor detects a predetermined increase in mouth pressure generated by the mammal.

Suitable pumps include positive displacement pumps or blowers, such as diaphragm pumps, plunger pumps, and gear pumps. Preferably said pump is a diaphragm pump, also called membrane pump.

In the above embodiment and others disclosed herein, said pump or fan is adapted for maintaining a predetermined flow within the interval of about 1 to about 1000 ml/s, or about 5 to about 600 ml/s, with an accuracy of at least +/−10% of the desired value. Other preferred intervals are for example 1-100 ml/s, 20-350 ml/s, 40-400 ml/s, 40-600 ml/s and different sub-intervals thereof.

In a preferred embodiment, freely combinable with the other embodiments, said predetermined flow is flexible, in the sense that multiple flow rates can be maintained, each within the interval of about 5 to about 400 ml/s. Preferably the flow is maintained at or around at least one predetermined flow with an accuracy of +/−10%. More preferably each flow is maintained with an accuracy of at least +/−5% of the desired value.

Further, the device or system is preferably adapted to eliminate the contribution of nasal air by securing velum closure by maintaining a mouth pressure of at least 3 mbar, preferably within an interval of about 3 to about 40 mbar.

In the here disclosed methods and devices, the component to be detected/determined in exhaled air can be chosen from a gaseous component such as carbon monoxide, oxygen and nitric oxide, to mention three non-limiting examples; particulate matter such as for example cells, microbes, and macromolecules; and volatile organic compounds, such as drug metabolites, disease markers etc.

According to a preferred embodiment, freely combinable with the other embodiments presented herein, this component is nitric oxide and said sensor specific for the component to be determined is a nitric oxide sensor chosen from colorimetric, ultrasonic, chemiluminescence and electrochemical nitric oxide sensors.

According to an embodiment freely combinable with any other embodiment disclosed herein, the device or system further comprising means for storing a sample, such as a buffer chamber, a flexible container, a bellows or a cylinder and piston arrangement, or at least a port to which a separate storage container can be attached.

According to an embodiment freely combinable with any other embodiment disclosed herein the device or system is adapted for being connected to a second device for analysing one or more components in a sample of exhaled air. The device or system is adapted for being connected upstream of said second device, i.e. between an exhaling mammal and said second device. Alternatively, the device or system is adapted for being connected downstream of said second device.

In contrast to PAP ventilation, the device and method according to embodiments of the invention is coupled to the exhalation, not the inhalation, and it is the mammal, not the device, that delivers the positive pressure.

### BRIEF DESCRIPTION OF THE DRAWINGS

The invention will be described in closer detail in the following description, non-limiting embodiments, examples, and claims, with reference to the attached drawings in which:

FIG. 1 is a graph illustrating the concept of an exhalation profile, showing the exhalation flow rate (Q, ml/s, a thick, continuous line), the mouth pressure (P_{M}, mbar, a thin, dotted line), and the accumulated volume of exhaled breath (V, I, dashed-dotted line) as the function of time (t, s) during an exhalation where the flow rate is kept practically constant at 200 ml/s by a device or system according to an embodiment of the invention;

FIG. 2 is a graph showing the flow, pressure and concentration of NO as a function of time (t, s) during an exhalation where the flow is adjusted at two different, and predetermined flow rates, Q₁ and Q₂, by a device or system according to an embodiment of the invention. The exhalation flow rate and mouth pressure are indicated as in the previous graph and the concentration of endogenous NO (NO, ppb,) is shown as a dashed-dotted line, exhibiting two plateaus, NO, Q₁ and NO, Q₂;

FIG. 3 is a graph illustrating an embodiment of a method enabling fractionated sample collection during one exhalation, or if repeated, during tidal breathing. The exhalation flow rate, mouth pressure, accumulated volume of exhaled breath are indicated as in FIG. 1. The abbreviations V_{W}, V_{S}; and V_{T} indicate the dead space volume which is discarded, the sample volume and the total exhaled volume, respectively.

FIG. 4 is a graph illustrating an embodiment of a method enabling fractionated sample collection during tidal breathing, here shown as three consecutive exhalations. The exhalation flow rate, mouth pressure, and accumulated volume of exhaled breath are indicated as in FIG. 1. A sample can be taken at any point in time during the exhalation, and by taking a fraction of the exhaled air at the same time point during each exhalation, many small samples can be pooled;

FIG. 5 schematically shows components and functions of a device according to an embodiment of the invention;

FIG. 6 schematically shows components and functions of a device according to an embodiment of the invention, including means for separate regulation of the flow in two flow intervals, here exemplified as Q≦100 ml/s and Q≧100 ml/s, respectively;

FIG. 7 schematically shows an embodiment where a flow generator (B) is connected downstream of a device (A) for taking and optionally analyzing a sample of exhaled air, and a user interface (C) here shown as a personal computer, where the device for taking a sample controls the function of the flow generator, via the personal computer;

FIG. 8 schematically shows an embodiment similar to that of FIG. 7, but where the device D is adapted to collect a sample or pool a number of samples in a sample container for offline measurement.

FIG. 9 schematically shows an embodiment where a device A as in FIG. 7 is connected to a flow generator B, where the flow generator B is controlled by the device A, so that an exhalation flow is initiated and maintained by the flow generator B only when a minimum mouth pressure is detected by the device A.

FIG. 10 schematically shows an embodiment where the device A is connected to a flow generator B, a set-up which is useful for example when a flow generator is used to aspirate a sample from the nasal cavity of a subject. In such embodiments, device A can control device B, as indicated by the arrow. Further, the subject may optionally blow into a mouthpiece, MP, having an orifice ensuring a mouth pressure sufficient to close the soft palate. In the alternative, the subject can be asked to take a deep breath, and to hold their breath during the aspiration of a sample from the nasal cavity.

FIG. 11 schematically shows an embodiment where a flow generator B is connected downstream of a device D for taking a sample, for example by collecting a sample in sample container 30, wherein said sample can be analyzed in a separate device E, and where the flow generator is controlled by the device for taking a sample.

### DESCRIPTION OF EMBODIMENTS

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

Also, the term “about” is used to indicate a deviation of +/−10% of the given value, preferably +/−5%, and most preferably +/−2% of the numeric values, where applicable.

In addition to the above, the following terms will be used:

The term “subject” is intended to encompass both human and animal subjects, and both healthy and diseased, conscious and unconscious.

The term “sample” encompasses all types of samples that can be extracted from exhaled breath, such as gaseous components such as carbon monoxide and nitric oxide to mention only two non-limiting examples, particulate matter such as for example cells, microbes, and macromolecules; and volatile organic compounds. Other parameters that preferably are measured include temperature and humidity, which can be used as such, or in combination with other measured properties, for example to correlate a measured parameter to the temperature or humidity, at which it was measured.

The terms “test” and “diagnosis” are intended to include clinical and veterinary applications such as the diagnosis and monitoring of pathologies in humans and other mammals, as well as in non-clinical research, preventive health care and exercise.

The term “infant” is intended to include human children ranging from newborn, including prematurely born babies, to children of about 24 months.

The term “small children” is intended to include human children ranging from the age of about 2 years to about 5 years.

The term “mouthpiece” is used to denote the element through which the mammal exhales into the device, creating an airtight seal between airways of the mammal and the device, and can include a short tube that goes in between the lips, and may include a flange that fits between the lips and the teeth and gums, and/or a soft flange that fits over the lips, or a mask, such as a half-mask or full face mask. The mouthpiece shown in FIG. 10 however illustrates a special case, where a separate mouthpiece with an orifice is used to create a mouth pressure, sufficient to close the soft palate and isolate the nasal airways during sampling from the nasal airways.

The term “system” is intended to include combinations of apparatuses and devices, including such known at the priority date, but assembled in combinations and performing functions disclosed for the first time in this application.

The term “pre-determined” as in “pre-determined value” means a numerical value assigned to a parameter, either as entered by the user or operator, e.g. a patient in a home setting, a nurse or a physician in a clinical setting. The term however encompasses values that follow from another value, for example entering the age, sex and weight of a subject may generate a “pre-determined” value for another parameter.

The term “incentive” encompasses any type of feed-back, which directs, supports and motivates a mammal to perform the required inhalation and exhalation maneuvers. The incentive in particular encourages and motivates the mammal to initiate the exhalation and to maintain a required minimum pressure or flow for the time needed for the sampling.

### Method

Constant Flow Embodiment

One embodiment of the method comprises the steps of detecting the beginning of an exhalation and preferably already the intent to exhale, as a predetermined increase in either mouth pressure or exhalation flow. A pressure sensor adapted to measure mouth pressure communicates the measured pressure to a unit which compares the measure value with the predetermined pressure required for generating flow. When the predetermined value is reached, said unit sends a signal which triggers the activation of means, such as a pump or a fan, for creating an exhalation flow, which then is kept substantially constant during the duration of the exhalation. When a predetermined volume has been exhaled, calculated as the duration of exhalation times the exhalation flow, a signal is sent which deactivates the means for creating a flow.

Similarly, an increase in flow can trigger the activation of means, such as a pump or a fan, for creating an exhalation flow, provided that a preset minimum mouth pressure is detected.

In order to avoid discomfort to the subject using the device, the exhalation flow will be automatically stopped if the subject stops exhaling and wishes to inhale. Accordingly, when a decrease in pressure is detected, indicative of the termination of the exhalation, the means for creating an exhalation flow are deactivated and the subject allowed to inhale.

One or more samples of the exhalation air can be taken during the exhalation when the flow is maintained at a constant level, preferably during the second half of the exhalation, or at least after a predetermined period of time or volume in order to exclude the air originating from the dead space in the airways of the subject.

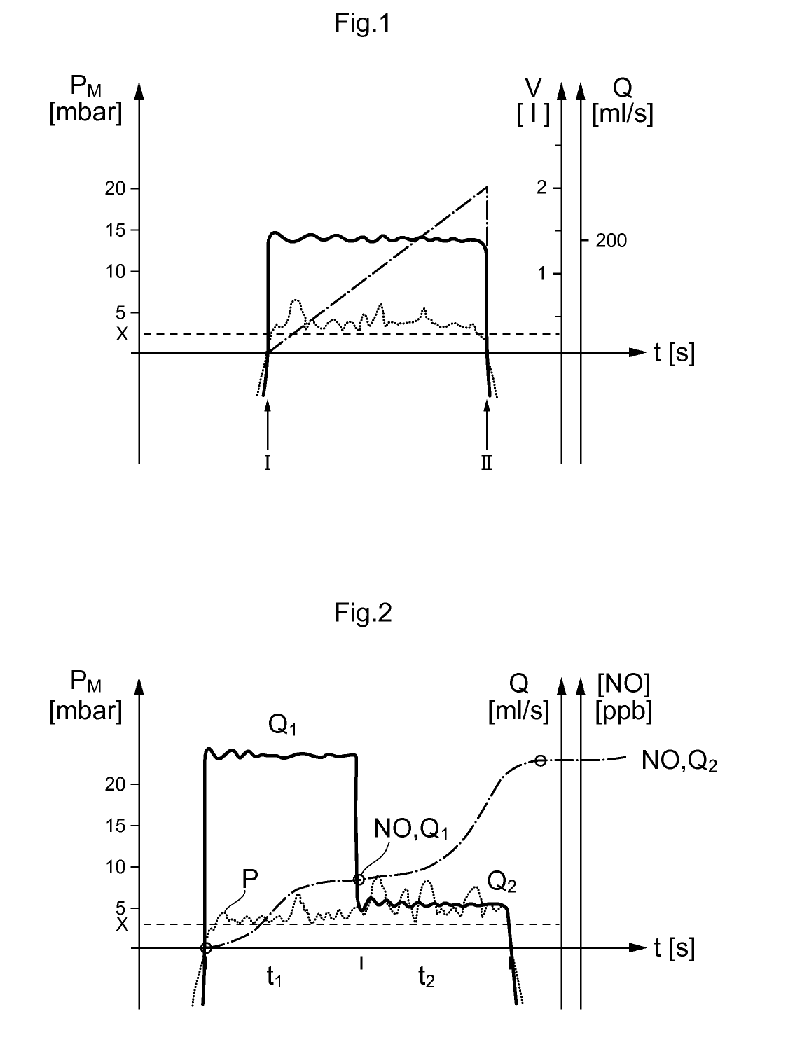

The above embodiment is illustrated in FIG. 1, which shows a graph where the mouth pressure (P_{M}, mbar), the exhaled volume (V, liter), and the exhalation flow (Q, ml/s) are shown on the three verticals, respectively. The horizontal axis illustrates time (t, s). Pressure is shown as a thin, dotted line, exhalation flow as a thick, continuous line, minimum mouth pressure is marked as “X” and shown as a horizontal, dashed line, and exhaled volume as a dot-dashed line.

In an exemplary embodiment, illustrated in FIG. 1, the spontaneous end of an inhalation and the beginning of an exhalation is shown as the point I where the mouth pressure curve goes from negative or zero to positive. When the mouth pressure generated by the subject reaches a predetermined threshold value “X”, for example about 0, 1, 2 or 3 mbar, means for generating an exhalation flow is/are activated. The exhalation flow then increases rapidly and is stabilized at the pre-set value. The means for creating an exhalation flow are then operated to maintain a predetermined flow, here exemplified as 200 ml/s.

Simultaneously the mouth pressure is monitored, in order to ensure that it remains over a set value, in order to close the velum and maintain it closed, so as to eliminate contamination from nasal airways. It is however possible, in the case of very low pressures, that a nose clip is used to exclude contamination from nasal air. As shown by the fluctuations of the thin, dotted line, the mouth pressure can vary, but will be maintained above the threshold value “X”. The device can be described as “forgiving” with regard to mouth pressure, as long as a minimum pressure is maintained. Even if the subject exhales forcefully, creating a higher mouth pressure than necessary to close the velum, the flow will be kept practically constant during the exhalation.

When the desired predetermined exhalation time or volume, here schematically shown as 2 liters, corresponding to 10 sec at a flow of 200 ml/s, is reached, the means for creating a flow is/are deactivated, and the flow ceases. Alternatively, when the subject spontaneously stops exhaling and prepares to inhale, this is detected as a decrease in mouth pressure, here shown in point II. This decrease can be detected as a sharp decrease in mouth pressure, e.g. a significant change in the derivate of the pressure curve, or as a negative mouth pressure, and thereby distinguished from possible variations in flow during the exhalation. When this decrease in mouth pressure is detected, the means for creating an exhalation flow is/are deactivated, and the mammal allowed to inhale. The above steps can be repeated during several breathing cycles, in order to allow fractionated sampling during tidal breathing.

Multiple Flow Embodiment

Another embodiment of the method involves the creation of an artificial exhalation profile, preferably comprising multiple sequential flow values, during one exhalation. It is also conceived, due to the limited length of one exhalation, that the exhalation maneuver is repeated, and that the flow is controlled at one or more preset values during each exhalation, thus ensuring that samples can be taken from exhalation air originating from different sections of the airways.

The methods according to these embodiments also comprise the steps of detecting the intent to exhale, as a predetermined increase in either mouth pressure generated by the mammal or increased flow of exhaled air. Preferably it is an increase in mouth pressure that is chosen. This triggers the activation of means for creating an exhalation flow, which then is adjusted to two or more substantially constant flow values, each for a predetermined time or volume. When a predetermined time has passed, a desired volume collected, or when a decrease in mouth pressure is detected, indicative of the termination of the exhalation, the means for creating an exhalation flow are deactivated and the mammal is allowed to inhale. One or more samples of the exhalation air can be taken during the exhalation when the flow is maintained at a constant level, at different flows, and preferably during the second half of the exhalation, or at least after a predetermined period of time or volume, in order to exclude the air originating from the dead space.

This embodiment is illustrated in FIG. 2, which shows a graph where the mouth pressure (P_{M}, mbar), the exhalation flow (Q, ml/s) and NO concentration (ppb) are shown on the vertical axis. The horizontal axis represents time (t, s). The mouth pressure is shown as a thin, dotted line, the minimal required mouth pressure as a horizontal, dashed line, and exhalation flow rate as a thick, continuous line.

The concentration of endogenous NO is shown as a dashed-dotted line, exhibiting two plateaus, one representative of the different flows, here indicated as NO, Q1, which is the NO plateau at Q1, and NO, Q2, the NO plateau at Q2.

In an exemplary embodiment, the spontaneous end of an inhalation and the beginning of an exhalation is where the slope of the pressure curve goes from negative or zero to positive. When the pressure reaches a predetermined threshold value, for example 0, 1, 2 or 3 mbar, the means for creating a flow is/are activated. The means for creating a flow are then operated to maintain a predetermined first flow, Q1, here exemplified as 100 ml/s for a predetermined period of time, here indicated as t1. The flow is then decreased to another, preferably significantly lower flow, Q2, here illustrated as 20 ml/s for a predetermined period of time, here indicated as t2.

During this procedure, samples of exhaled air can be taken at different time points and exhalation flow rates, which make it possible to sample exhalation air originating from different parts of the airways including the lungs. It is however important that the time at each different flow value is sufficiently long to allow the exchange of gas from the airway walls, and to allow sample collection.

If desired, or if necessary in order to allow sampling at different flow rates, different points in time, and/or after the exhalation of a predetermined volume, the above can be repeated during several exhalations.

There are many advantages associated with the creation and maintenance of an exhalation flow rate independently of exhalation pressure, or triggered already by a very low active exhalation pressure. The methods and device can be characterized as “forgiving” with regard to how the exhalation is performed by the mammal. As long as a minimal mouth pressure is achieved, the device will regulate and maintain the exhalation flow. The subject enjoys maximum support and convenience, which encourages cooperation, even if only minimal cooperation is necessary. The active extraction of targeted fractions of the exhalation air guarantees high reproducibility and accuracy.

Further, the device and methods according to embodiments of the invention allow the mammal to control the breathing pattern to a large extent, which also increases convenience and supports cooperation and compliance.

As explained above, the method is applicable to the sampling of different analytes know or suspected to be found in exhaled air from a mammal. A sample of the exhaled air is accordingly brought into contact with a suitable sensor. The accurate control of the exhalation flow may be significant for the determination of any analyte in exhaled air, as controlling the flow increases accuracy, guarantees repeatability, and makes it possible to associate the analyte to different portions of the airways, as well as distinguishing between analytes having different diffusion characteristics, i.e. which pass from the airway tissues into the flow of air in the airways with different speeds.

The sample can be led directly to a sensor or other means for determining the concentration of the analyte or substance/substances of interest, or alternatively stored temporarily in one or more substantially gas-tight and inert container or containers, such as a Mylar® bag, possible to detach from the device. The use of such containers makes it possible to collect multiple samples and analyze them separately.

Currently, one important application is the analysis of exhaled, endogenous nitric oxide (NO). The concentration of nitric oxide, detectable in ppb levels in exhaled air, can be using different techniques, for example by colorimetric analysis, chemiluminescence, electrochemical sensors, thin film technologies and immobilized chemical reactants, etc. When determining the concentration of NO, detectable in ppb levels, accurate control of the exhalation flow becomes particularly important, as in the determination of any diagnostically relevant analyte. In the determination of NO as well as in other diagnostic applications, compliance and repeatability are particularly important. For NO in particular, it is of considerable relevance to be able to associate the analyte to different portions of the airways. This allows the investigation of NO specific inflammatory processes in the central airways, the bronchial tree and even the peripheral lung.

An embodiment, freely combinable with the above disclosed embodiments, is thus the application of the methods and devices to the analysis of nitric oxide in samples of exhaled air obtained through methods as disclosed here. By way of example, one or more samples can be drawn from the exhaled air during constant flow and at a known point in time, and subjected to analysis of the NO concentration which is in the range of 1-600 ppb for orally exhaled air, and 5-6000 ppb for nasally exhaled air (these ranges dictate the required sensitivity of the sensor). In one embodiment, the NO value is recorded together with the corresponding exhalation flow, during which the sample was taken. The values obtained are recalculated to NO values at a flow of 50 ml/s with an accuracy of +/−10%. Optionally also the time, mouth pressure and exhaled volume is recorded.

The method is applicable also to embodiments where a sample is taken, but not immediately analyzed. Instead, samples can be taken and stored in one or more separate containers for later analysis. Such containers should be made of a material that does not react with components of the sample, in particular with the component(-s) to be determined. The material such also not emit the component that is to be determined, or emit any substance(-s) that could react therewith or otherwise influence the analysis. One example of suitable sample containers are containers made of polyester film, for example polyethylene terephtalate films, such as Mylar® films.

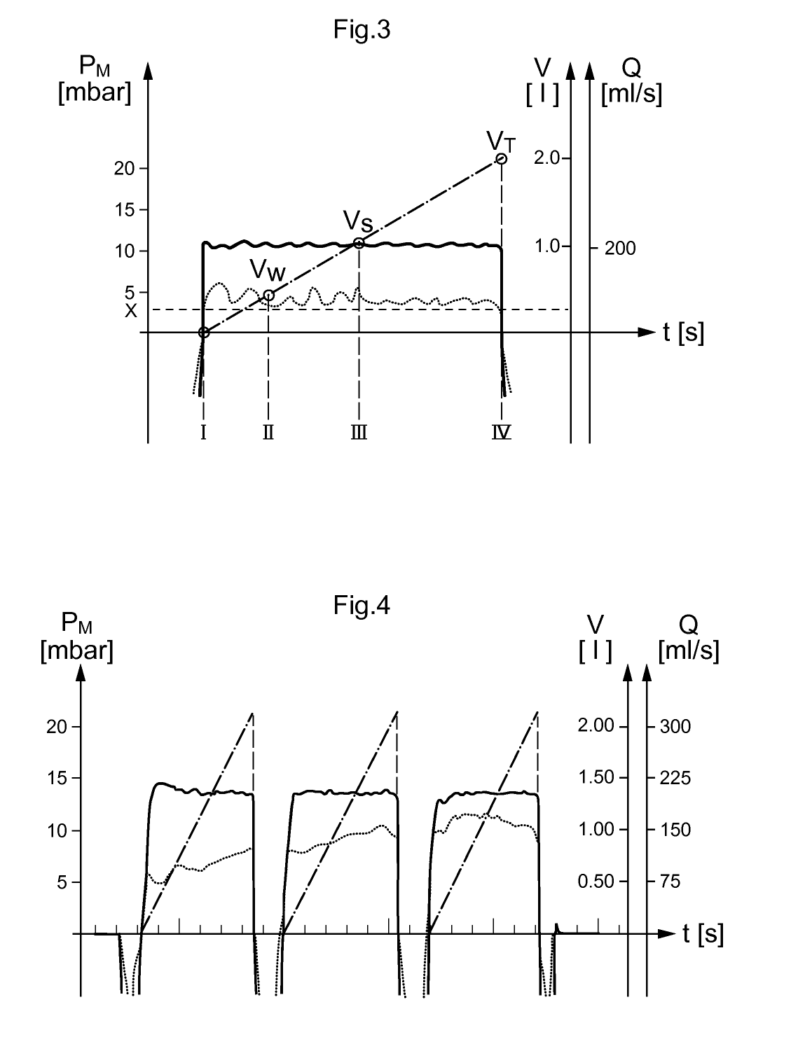

FIG. 3 is a graph showing the fractionated extraction of a sample during one exhalation at constant flow. In the graph, mouth pressure (P_{M}, mbar), the exhaled volume (V, liter), and the exhalation flow (Q, ml/s) are shown on the three verticals, respectively. The horizontal axis illustrates time (t, s). Mouth pressure is shown as a thin, dotted line, flow rate as a thick, solid line, minimum mouth pressure “X” as a dashed line, and the cumulated exhaled volume as a dashed-and-dotted line.

As from point I, the flow is maintained at a substantially constant level, here exemplified as 200 ml/s. During a first period of exhalation between I and II, the exhaled air is led to the environment and a volume V_{w} is discarded. This may serve to discard the dead space, for example the parts of the airways which do not participate in the gas exchange, or which can be contaminated by NO of nasal origin, bacterial origin or the like.

Between points II and III, a sample is collected, having the volume V_{S} (V_{S}=V_{III}−V_{W}) The controlled exhalation is interrupted at time point IV, and the subject allowed to breath freely when either an intent to inhale is detected, or a preset exhaled volume has been reached. The remaining volume after the sampling, until the pre-set total volume V_{T} is reached, i.e. during the time period III to IV, may also be discarded. This procedure can be repeated during several exhalations, for example in tidal breathing, and allows the collection of multiple samples from a well defined fraction of exhalation air.

FIG. 4 illustrates an embodiment of a method enabling fractionated sample collection during tidal breathing, here shown as three consecutive exhalations. The exhalation flow rate, mouth pressure, and accumulated volume of exhaled breath are indicated as in FIG. 1. Each breath is controlled to a flow rate of about 200 ml/s, and simultaneously the mouth pressure is maintained or allowed to vary between about 5 and about 10 mbar. During a time period of 10 seconds, a volume of 2 liters is exhaled, allowing the dead space to be discarded, and the taking of one or more sample(s) representing different fractions of exhaled air.

Nasal Sampling

Further embodiments of the invention relate inter alia to sampling from other air filled cavities, such the nasal airways. One embodiment relates to the examination of disorders of the nasal airways, wherein a sample is aspirated from the nasal airways while the velum or soft palate is kept closed, in order to isolate the oral cavity and the lower airways. A significant advantage of the methods and devices is that the flow rate can be adapted to the sample to be taken, as well as to reduce discomfort to the patient.

In one embodiment, a sample is aspirated from the nasal airways at a flow rate of about 1-100 ml/s, or different sub-intervals thereof, preferably within about 10-50 ml/s while the velum is kept closed.

The level of nasal NO is known to be altered in several nasal disorders. A reduced nasal NO concentration has been shown in different disorders affecting the paranasal sinuses, such as chronic rhinosinusitis with or without nasal polyposis, and acute sinusitis, and correct treatment may partly or entirely restore the nasal NO levels. Reduced NO levels have also been described in patients with cystic fibrosis, and most markedly in patients with primary ciliary dyskinesia. Increased nasal NO levels have been shown in patients with allergic rhinitis. With the commonly used methods to date, nasal NO measurement has been difficult to be applied to small children, due to the long sampling times.”

It is contemplated that that specific markers, such as nasal NO, in particular when the sample is taken with a device allowing a variation of the flow rate, and/or the sampling at different flows, can be useful in the investigation of various upper airway disorders.

These embodiments are discussed in closer detail below, in relation to the figures and examples, and illustrated by the determination of nasal NO as a screening step in the investigation of possible primary ciliary dyskinesia.

### Device

According to a another embodiment, the invention makes available a device or system for creating an artificial exhalation profile, comprising an inlet for receiving exhaled air from said mammal, a flow channel, a pressure sensor, a flow sensor, a control unit, and a pump or fan for creating an exhalation flow, wherein said pressure sensor and/or flow sensor are adapted to supply signals to the control unit, and the control unit controls the pump or fan in such manner, that said pump or fan is activated when said pressure and/or flow sensor detects a predetermined increase in mouth pressure and/or flow, and deactivated when said pressure sensor detects a predetermined decrease in mouth pressure.

According to yet another embodiment, the invention makes available a device or system for taking a sample of exhaled air from a mammal, comprising an inlet for receiving exhaled air from said mammal, a flow channel, a pressure sensor, a flow sensor, a control unit, and a pump or fan for creating an exhalation flow, wherein said pressure sensor and/or flow sensor are adapted to supply signals to the control unit, and the control unit controls the pump or fan in such manner, that said pump or fan is activated when said pressure and/or flow sensor detects a predetermined increase in mouth pressure and/or flow, and deactivated when said pressure sensor detects a predetermined decrease in mouth pressure.

A device according to the above embodiment and variants thereof can be termed “a flow generator” and this device can be a separate or integrated part of a system comprising one or more means for measuring mouth pressure and flow and optionally other parameters or it can comprise these means or functions in itself.

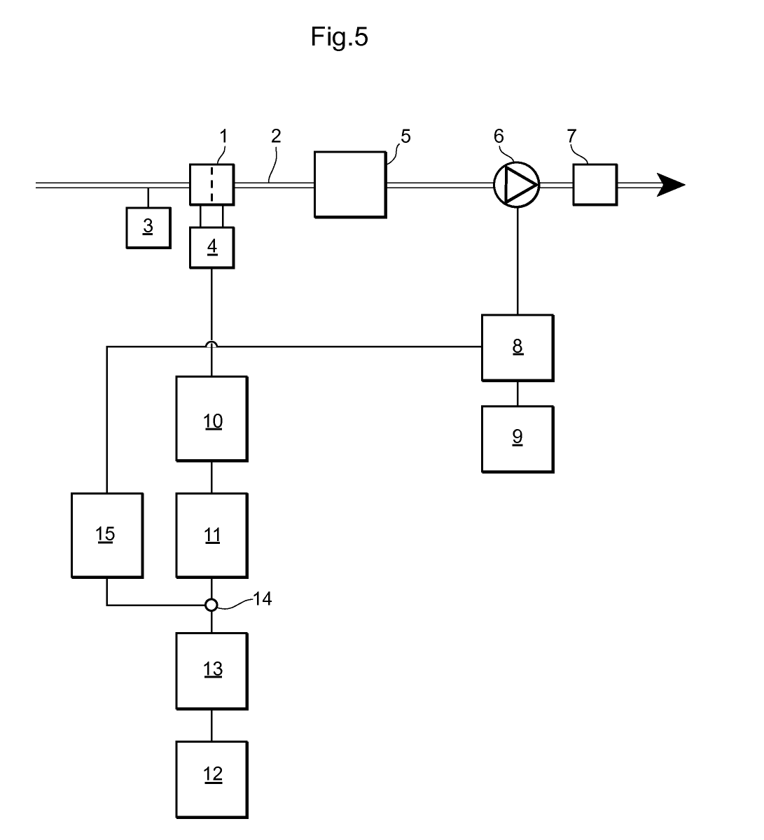

One embodiment of such a flow generator is illustrated in FIG. 5, where a system is schematically shown including a pneumatic resistor 1 in a flow channel 2, wherein said pneumatic resistor 1 is connected to a differential pressure transducer 4 for measuring flow. Further, the system includes a pressure sensor 3 for measuring mouth pressure. Downstream, a buffer 5, a pump 6, and optionally a noise reducer 7 are located in the flow channel 2.

The pump 6 is connected to a power control 8 and a power supply 9, controlled by a control circuit including a flow linearization function 10 receiving signals from the pressure transducer 4 and sending signals to a transfer function 11. The system also includes an input function 12 where desired parameters such as mouth pressure, flow, duration and total volume can be entered. The input function communicates with a transfer function 13 which recalculates the parameters as reference values to be compared to measured values in the equilibration point 14. Depending on the relation between the reference values and the measured values, a signal is sent to a regulator 15 which controls the pump 6 via the power control 8.

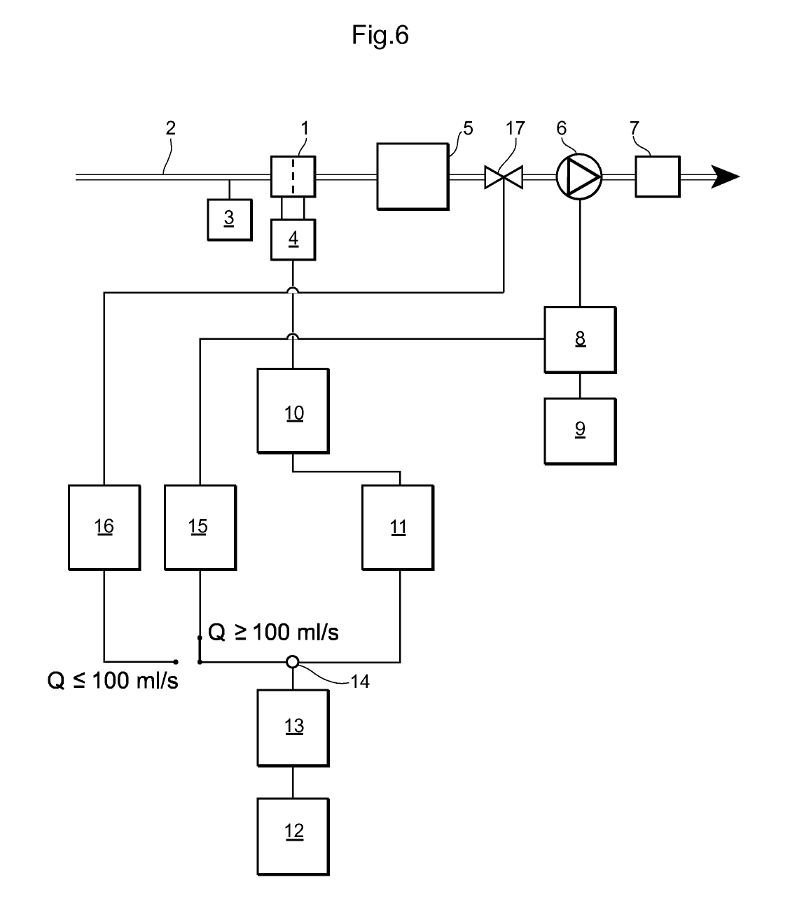

FIG. 6 illustrates an embodiment where, in addition to the features shown in FIG. 5, the flow generator includes a second regulator 16 and a valve or valve block 17. At low flows, here exemplified as a flow of less than about 100 ml/s, the regulator 16 controls a valve 17 positioned in the flow channel 2. At low flows, the pump is operated at constant speed, and the flow is controlled by regulating the aperture of the flow channel by regulating the valve 17. At high flows, here exemplified as a flow of more than about 100 ml/s, the valve 17 is set in an open position, and the frequency or capacity of the pump 6 is controlled by regulator 15.

In an embodiment (not shown) the flow channel is divided into several parallel channels, and the valve block 17 is part of a manifold of two or more valves, each positioned in a channel with a specific diameter. The closing of all valves but one will force the exhalation air through the remaining channel, and the diameter of the chosen channel will aid in controlling the flow. It is conceived that in addition to forcing the exhalation flow to pass through channels of different diameter, the diameter of the channel could also be varied by the operation of servo controlled valves or the like.

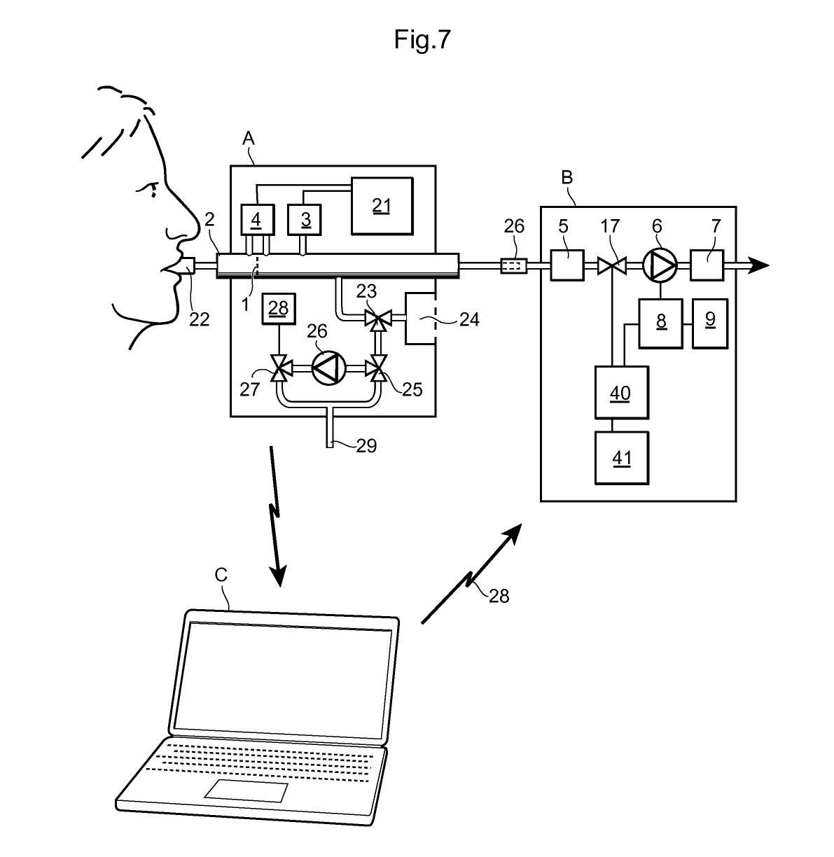

FIG. 7 illustrates an embodiment where a device A for taking a sample of exhaled air is connected to a flow generator B. Both devices communicate with a user interface, for example a personal computer C. The device A for taking a sample of exhaled air comprises a flow channel 2, a pneumatic resistor 1 in said flow channel, a differential pressure transducer 4, and a pressure sensor 3 for measuring mouth pressure. A subject exhales into the flow channel 2 through a mouthpiece 22. The device A further comprises a control unit 21 for receiving signals from the pressure transducer 4 and pressure sensor 3, and adapted to controlling a valve 23. When the subject inhales, air can be drawn through a filter or scrubber 24, removing or significantly reducing the concentration of the component to be determined from the inhalation air.

When the subject exhales, and the flow and pressure are within a pre-determined interval, the valve 23 can be opened to take a sample. A pump 26 controls the flow of the sample, and using adjustable valves 25 and 27, a sample can be fed to a sensor 28. Through valve 25, a sample of exhaled air can also or alternatively be led into a sample container (not shown), connected to the device A via gas-tight coupling 29. The sample container can be a sample bag or other container, suitable for the volume of sample and type of component in exhaled air that is the focus of interest.

Further, the device A is connected to the flow generator B through a gas-tight coupling, here illustrated as 26. The flow generator B comprises a buffer 5, a pump 6, and optionally a noise reducer 7. The pump 6 is connected to a power control 8 and a power supply 9, controlled by a control circuit 40, which also controls a valve 17, positioned in the flow channel. The control circuit 40 has an interface 41, capable of receiving and optionally also sending signals to a user interface, here illustrated as a computer C.

Reference values and other data, for example patient data, can be entered via the computer C. In operation, the computer C exchanges signals with the device A, for example the flow and mouth pressure measured by the differential pressure transducer 4 and the pressure sensor 3 for measuring mouth pressure. The computer C also communicates with flow generator B, sending signals, for example the reference values for flow, the measured flow, start and stop signals, etc. The signals can be transmitted through a wireless connection (such as Bluetooth, WiFi, IR etc) or through a wired connection (such as USB).

The flow generator B can also be controlled directly by the device A, for example in such a fashion that device A, when an increase in mouth pressure is detected, sends a signal to B which initiates the operation of the pump 6. The operation of the pump 6 and the valve 17 may then be controlled by A, based on the measured mouth pressure and flow.

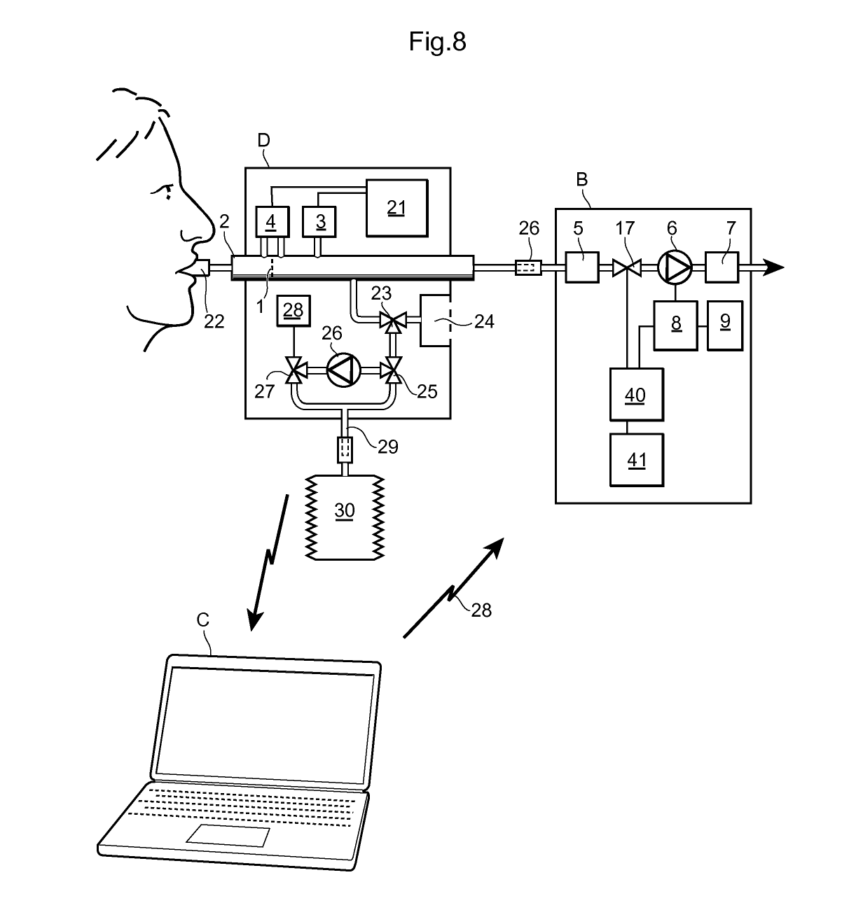

FIG. 8 shows an alternative or modification of the embodiment shown in FIG. 7, where the device, here denoted D, is adapted to collect a sample or pool a number of samples in a sample container for off-line measurement. Device D supplies a sample through a gas tight connection 29 to a separate sample container 30, for off-line measurement.

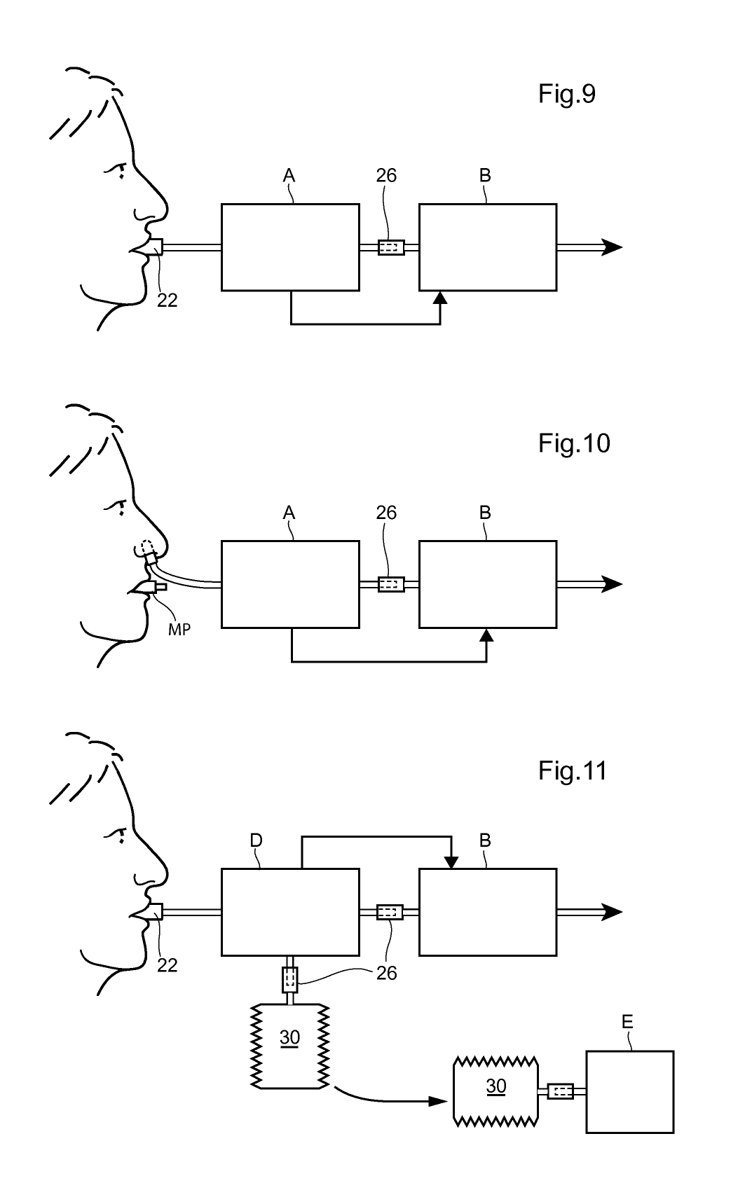

FIGS. 9, 10 and 11 schematically show three different and non-exclusive embodiments. FIG. 9 shows an embodiment where a flow generator B is placed downstream of a device A, connected thereto with an airtight connection 26. In this fashion, the flow generator “pulls” the exhalation air through the device A, ensuring a constant flow or different known flows during the sampling and analysis performed by the device A. Here, a subject exhales into the device A through a mouth piece 22, and A measures exhalation parameters, such as flow and mouth pressure, and controls the flow generator, as indicated by the arrow.

FIG. 10 shows an embodiment where the flow generator B for creating a flow is connected downstream to a device A through an airtight connection 26. Preferably said connection 26 is detachable, allowing the separate use of each device, as well as the attachment of different devices (not shown) to the flow generator B. The flow generator is here shown with a tube fitted in the nose of a subject for aspirating a sample of nasal air. This merely serves as an example of an application of the device to a situation where a sample needs to be aspirated, where it is difficult or impossible for the subject to exhale or otherwise supply a sample.

Further, the subject may optionally blow into a mouthpiece, shown as “MP” in FIG. 10, having an orifice ensuring a mouth pressure sufficient to close the soft palate. In the alternative, the subject can be asked to take a deep breath, and to hold the breath during the aspiration of a sample from the nasal cavity.

Nasal sampling may be more prone to irregularities as the nasal airways can be obstructed, the sampling tube/nose olive can be improperly inserted or shifted during the procedure. Therefore, in one embodiment of the device and method for taking a sample of air from the nasal cavity, the performance of the procedure can be improved by calculating the output per time unit of the marker. Using NO as an example, it can be stipulated that the NO concentration exhibit an inverse linear relationship to the sampling flow, when using flows within the preferred flow range (10-50 ml/s). Thus, by calculating NO output (NO concentration times flow), variations in sample flow rate are adjusted for. NO output can typically be expressed as picol/s or nanol/min.

The embodiment illustrated in FIG. 10 reflects devices for the examination of disorders of the nasal airways, as well as methods for this purpose, such as a method for the screening of patients suspected of having primary ciliary dyskinesia (PCD). PCD is a genetic disorder manifesting itself at an early age, but frequently not properly diagnosed until many years later. The embodiments presented herein offer a possibility to obtain an early diagnosis, or at least to rule out the possibility of PCD, as the high levels of endocrine NO encountered in nasal air from healthy, are absent in PCD patients.

In one embodiment, applicable to both devices and methods for aspirating a sample from the nasal airways, it is conceived that a tube with a nasal adapter connected to a low-resistance filter, suitable for removing the marker to be analyzed, is inserted into one nostril, to reduce or remove said marker from ambient air. When a sample is aspirated from the contralateral nostril, and in particular when a large volume and/or high flow sampling is used, ambient air will enter through said low-resistance filter. When the sampling aims at collecting macromolecules or other biological markers present in the nasal air, a particulate filter can be used. Similarly, when the marker to be analyzed is NO, a low-resistance NO scrubber is used. This advantageous when the device is used/the method is performed in areas where the background level of NO can vary depending on traffic, weather conditions etc, e.g. in densely populated urban areas.

FIG. 11 shows an embodiment where a flow generator B is connected downstream of a device D for taking a sample of exhaled air, for example by collecting one or more samples in a sample container 30. The device D measures exhalation parameters, such as flow and mouth pressure, and controls the flow generator B, as indicated by the arrow. The sample container 30 can then be detached and connected to a separate analyzer E for determining the concentration of a component of exhaled air. Airtight connections 26 allow the transfer of the sample without leakage or contamination from ambient air. These connections can be any conventional male/female connections, such as plug and socket, threaded connections or bayonet couplings.

One advantage of the embodiments presented herein, devices and methods alike, is that exhaled breath samples can be obtained in a controlled and standardized manner also from subjects or patients that otherwise would have difficulties to comply with required breathing maneuvers, such as infants, children, elderly, unconscious or diseased, as well as taking breath samples from animals. The devices and methods not only make measurement possible, proper exhalation is ensured by the device and methods. Further, the device can safely be handled by the subjects themselves, making it possible to monitor a disease also outside a clinic or hospital setting, e.g. at home or at the workplace.

Another advantage lies in that the feature of a variable flow rate, while securing velum closure, makes it possible to test and apply different flow rates for different patients. It is a significant advantage if the required exhalation time can be minimized, for example by using the optimal flow rate for reaching a plateau of the marker concentration. When investigating infants, children and subjects with reduced lung capacity, a higher flow rate makes it possible to reduce the exhalation time required.

These advantages, and others that will be obvious to a skilled person, are particularly important when diagnostically relevant components in exhaled air are determined, as these are frequently present in minor quantities only (for example endogenous NO which is detectable in ppb levels) and because the results form the basis for diagnosis and in many cases, also therapy.

### EXAMPLES

### Example 1

### Single Breath FeNO Measurement

In an experimental set-up, a nitric oxide analyzer (NIOX Vario®, Aerocrine AB, Solna, Sweden, a modified version of the NO Vario® from FILT Lungen- and Thoraxdiagnostik GmbH, Berlin, Germany) was connected upstream to a device or system according to an embodiment of the invention, here called a flow generator. Healthy volunteers were asked to perform the prescribed breathing maneuver, inhaling and exhaling through the NIOX Vario®. Pressure and flow was recorded. The graph shown in FIG. 1 is representative of the results obtained.

When the NIOX Vario® registered an increase in mouth pressure, it sent a signal to the flow generator and activated a pump for creating a flow of 50 ml/s which was maintained for the duration of the exhalation. Mouth pressure was about 5 mbar in this example. The subjects did not report any discomfort from using the device.

### Example 2

### Step Test FeNO Measurement

So called Step test FeNO measurements, i.e. the measurement of exhaled nitric oxide (NO) at different exhalation flow rates allow volume based calculation of the depth of the inflammation area down the bronchial tree. The results can be used for the evaluation of NO specific inflammation processes in the central airways and/or in the periphery of the lung. It is believed that more clinical investigations can be performed with the development and availability of technically reliable test equipment, resulting in an increase in the amount of data. This will most likely lead to the development of new applications of NO measurements. There is for example a potential to improve the selection and dosage of anti-inflammatory drugs, as well as evaluating the effect of treatment within specific areas of the airways and lungs.

The subject was asked to perform an animation controlled exhalation against a regulated resistor element, maintaining a mouth pressure in the interval of 7 to 20 mbar. The exhalation flow was controlled at 100 and 20 mL/sec, +−10%. During one single exhalation, the exhalation flow was maintained at 100 ml/s for about 12 s, and then reduced to 20 ml/s for an additional 12 s. This is illustrated in FIG. 2.

The results show that the flow quickly settled at the first and second flow levels, and that a positive mouth pressure could be maintained throughout the exhalation. The time of 12 s was also sufficient for the NO values to reach their respective plateaus. In the experiment illustrated in FIG. 2, the first NO value was 17.2 ppb, and the second 43.5 ppb, respectively.

### Example 3

### Sample Collection for Off-Line Measurement

In another example, test subjects were exhaling into a device or system according to an embodiment of the invention and samples were collected in separate containers, for example Mylar® bags, for temporary storage and later analysis in a NIOX Flex® nitric oxide analyzer (Aerocrine AB, Solna, Sweden). A sample of the exhaled breath was extracted as illustrated in FIG. 3 using a set-up corresponding to that schematically shown in FIG. 8 and FIG. 11.

### Example 4

### Tidal FeNO measurement

Tidal FeNO measurements of exhaled nitric oxide (NO) at flexible flow rates allow the investigation of the inflammation down the bronchial tree under tidal breathing conditions. The results can be used for the judgment of NO specific inflammation processes in the central airways and/or in the periphery of the lung.

In the experiments, the subjects were allowed to breath against a resistance creating a mouth pressure in the interval of 7-20 mbar, both exhaling and inhaling through a device as described herein. When inhaling, air was led from the ambient, through a NO scrubber and a patient filter, to the subject. When the subject exhales, the device detects the start of an exhalation maneuver and begins to measure the flow and based on the flow, also calculates the exhalation volume.

In the experiment illustrated in FIG. 4, the exhalation flow was set at 200 ml/s, and the volume 2.0 l. The sampling was initiated after 2.5 s or when a volume of 500 ml had been exhaled, in order to discard the dead space volume. During the remaining exhalation, sample collection was performed at a rate of 2 ml/s.

An audible and/or visual signal indicated when the sample collection was finished for each exhalation, prompting the subject to inhale again. If desired, audible and/or visible signals can be used for guiding the inhalation and exhalation of the subject, based on the measured inhalation and exhalation volumes. Three consecutive exhalations were performed, reaching a total exhalation volume of 6.0 l and a sample volume of 45 ml. The NO measurement was performed offline, after the collection phase.

### Example 5

### Multi Flow FeNO Measurements

Multiple measurements of exhaled nitric oxide (NO) at different flow rates, preferably at 100 and 300 ml/s, allow the calculation of the alveolar NO concentration and the NO output of the lung in picolitres/second. The device and methods according to embodiments presented herein offer a simple and flexible but yet very accurate approach and it is believed that this will lead to an increase research activity, data collection and the development of future diagnostic uses.

Multiple FeNO measurements of exhaled NO concentration were performed under defined conditions. The mouth pressure was kept in the range of 7-20 mbar in order to guarantee velum closure. The flow rate was adjusted to 100 and 300 mL/sec+/−10% and four measurements made at each flow rate. The measured NO concentration was presented as NO output, picoL/s. The results are shown in Table 1.

TABLE 1

| Exhalation flow | NO concentration |
|---|---|
| (ml/s) | (ppb) |
| 106 | 12.3 |
| 105 | 12.1 |
| 108 | 11.5 |
| 109 | 11.9 |
| 289 | 6.4 |
| 289 | 5.7 |
| 289 | 6.1 |
| 293 | 5.8 |

It was found that the flow could be kept very accurately at the defined level during four repeated measurements. When plotting the results, the NO output can be described as with the formula:

Y=984.5+2.61−X

wherein Y is the NO output expressed as picoL/s and X is the flow, expressed as ml/s.

### Example 6

### Free Tidal FeNO Measurements

It is possible to adapt the measurement to different subjects. For infants, tidal breathing without any control of mouth pressure and flow may be the only way to perform the test. The apparatus according to the invention makes it possible to control and record, or merely record the flow and pressure, and use this information for controlling the sampling and analysis.

In one experiment, a child was breathing into the device, without flow control and only minimal flow resistance. A flow ranging from about 200 to about 400 ml/s was recorded, and the mouth pressure remained low, about 1-2 mbar. The exhalation volume per breath was 1500 ml, and no part of the exhalation air discarded. A sample was extracted at the rate of 2 ml/s.

After ten consecutive breaths, a total exhaled volume of 15.4 l had been recorded, and a sample volume of 1486 ml collected. The NO measurement was performed offline, after the collection phase.

### Example 7

### External NO Measurements

The device or systems disclosed herein can also be used for analyzing NO in any externally collected gaseous sample. This is sometimes referred to as off-line measurement, and includes the handling of gas samples generated in clinical trials or under experimental conditions. In some applications, this involves the measurement of NO values in the 5-500 ppb range, but NO values can be in the range of 5-6000 ppb in samples collected from the nasal cavity or the sinuses.

An external sample, including pooled samples, can be delivered in gas-tight and inert container or containers, such as Mylar® bags, flexible thermoplastic bags or balloons, syringes or the like. The container is then connected to a device according to an embodiment of the invention, and the sample led to the sensor at a defined flow. It is an advantage that the flow can be controlled, as in the embodiments of the invention, as this ensures good repeatability and accuracy.

### Example 8

### Nasal NO Measurement with External Flow Generator

The measurement of nasal nitric oxide (NO) has gained increasing interest as a method for the screening of certain diseases, in particular a genetic disorder known as primary ciliary dyskinesia.

The measurement of the NO concentration in the nasal cavity of human test subjects was tested under defined conditions. The mouth pressure was kept in the range of 7-20 mbar in order to guarantee velum closure. The subject was asked to perform an animation controlled exhalation against a resistor element to keep the mouth pressure in the defined range. A flexible tube with a soft silicone nasal adapter was inserted in one nostril of the subject, and connected to the sample pump of a modified NIOX Mino® (Aerocrine AB, Solna, Sweden). The sample pump fed the sample to the electrochemical sensor. The flow rate of orally exhaled air was about 50 ml/s, where as the nasal flow rate was 2 ml/s.

Nasal NO was investigated using a NIOX Vario® (Aerocrine AB, Solna, Sweden). The subject was required to exhale against a resistor element, for example a tube ending in a small orifice (illustrated as “MP” in FIG. 10), maintaining a flow of about 50 ml/s and a positive mouth pressure in order to close the velum. A flexible tube with a soft silicone nasal adapter was inserted in one nostril of the subject, and connected via an adapter to the inlet of a flow generator, aspirating air from the nose at a flow rate of 200 ml/min for about 25 s, or 3000 ml/min for about 8 s.

In one measurement, where the mouth pressure was kept between 10 and 15 mbar, the flow of orally exhaled air was an almost constant 50 ml/s. Nasal air was collected for 25 s, during which time a clear NO plateau was reached. In this experiment, a concentration of nasal NO of 2275 ppb was recorded.

In another measurement, nasal NO was again investigated using a NIOX Vario® (Aerocrine AB, Solna, Sweden). The subject was however asked to take a deep breath and subsequently hold the breath in order to close the velum. A flexible tube with a soft silicone nasal adapter was inserted in one nostril of the subject, and connected via an adapter to the inlet of a flow generator, aspirating air from the nose during 10 s. Aspirating air at a flow rate of 20 ml/s resulted in a NO concentration of 60 ppb, and at 30 ml/s 50 ppb was recorded. A plateau NO concentration was reached after 4-6 s of aspiration, thus much faster compared to sampling with low nasal flow. It was also seen that the reproducibility was significantly better at higher flow rates.

There is yet no standard guideline definition available for nasal NO measurements. It is an advantage of the device and methods according to embodiments presented herein, that different nasal flow rates can be applied, and used to help define future standards.

Although the invention has been described with regard to its preferred embodiments, which constitute the best mode presently known to the inventors, it should be understood that various changes and modifications as would be obvious to one having the ordinary skill in this art may be made without departing from the scope of the invention as set forth in the claims appended hereto.

### CITED REFERENCES

### Patent Documents

JP Kokai 1998-048206 (Appl. No. 1996-216653)—SATO, Katsuhiko
U.S. Pat. No. 6,067,983—STENZLER, Alex
WO 93/05709—GUSTAFSSON, Lars E.
WO 95/02181—ALVING, Kjell et al
WO 2002/091921—MOILANEN, Eeva et al.
WO 2004/023997—VON BAHR, Pontus et al.
WO 2006/086323—NASON, Kevin et al.
WO 2008/106961—EICHLER, Ruediger et al.
WO 2011/038726—EICHLER, Ruediger

### Non-Patent Literature

American Thoracic Society, Medical Section of the American Lung Association: Recommendations for standardized procedures for the online and offline measurement of exhaled lower respiratory nitric oxide and nasal nitric oxide in adults and children-1999, in Am J Respir Crit Care Med, 1999; 160: 2104-2117
American Thoracic Society: An Official ATS Clinical Practice Guideline: Interpretation of Exhaled Nitric Oxide Levels (FeNO) for Clinical Applications, Am. J. Respir. Crit. Care Med. 2011 184: 602-615
Barroso N. C. et al., Exhaled Nitric Oxide in Children: A Noninvasive Marker of Airway Inflammation, Arch Bronconeumol. 2008; 44(1): 41-51 ERS Task Force Report 10: 1683-1693
Horwath et al., Exhaled breath condensate: methodological recommendations and unresolved questions, in Eur Respir J, 2005, September; 26(3):523-48
Wildhaber J. H. et al., Measurements of Exhaled Nitric Oxide with the Single-Breath Technique and Positive Expiratory Pressure in InfantsAm J Respir Crit Care Med Vol 159. pp 74-78, 1999

What is claimed is:

1. A method in the sampling of exhaled breath from a mammal, wherein said mammal exhales into a system comprising a flow channel, a pressure sensor, a flow sensor, a control unit, and a pump or fan for creating an exhalation flow, wherein
the pressure sensor measures pressure in the mouth of the mammal;
the flow sensor measures exhalation air flow in said flow channel;
the control unit receives signals from both the pressure sensor and the flow sensor, and sends a signal to activate said pump or fan for creating an exhalation flow when the measured mouth pressure exceeds a predetermined value, or when said pressure sensor and/or said flow sensor detects a pre-determined increase in pressure or flow;
said pump or fan for creating a flow, when activated, maintains a targeted exhalation flow at least at one pre-determined flow value, substantially independent of exhalation pressure; and
said pump or fan is deactivated when a predetermined time has lapsed, or when a predetermined exhalation volume has been exhaled, or when the pressure sensor detects a pre-determined decrease in mouth pressure.

2. The method according to claim 1, wherein the pressure sensor activates said pump or fan when said pressure sensor detects a pre-determined increase in mouth pressure generated by the mammal.

3. The method according to claim 1, wherein the exhalation flow rate is maintained at or around at least one value within the interval of about 1 to about 1000 ml/s.

4. The method according to claim 1, wherein the exhalation flow rate is maintained at or around at least one value within the interval of about 5 to about 600 ml/s, or about 5 to about 400 ml/s, with an accuracy of at least +/−10% of desired value.

5. The method according to claim 1, wherein mouth pressure is maintained at 3-40 mbar.

6. The method according to claim 1, wherein said exhalation flow is maintained at two or more sequential and pre-determined flow rates, each for a predetermined period of time or volume.

7. The method according to claim 1, further including the presentation of feed-back constituting an incentive for the mammal to initiate and maintain exhalation into the device.

8. The method according to claim 1, wherein said mammal is a human.

9. The method according to claim 1, wherein said mammal is a human chosen from the group including infants, small children, elderly, demented, mentally or physically disabled, and healthy.

10. A method for creating an artificial exhalation profile, wherein a mammal exhales into a system comprising a flow channel, a pressure sensor, a flow sensor, a control unit, and a pump or fan for creating an exhalation flow, wherein
the pressure sensor measures pressure in the mouth of said mammal;
the flow sensor measures exhalation air flow in said flow channel;
the control unit receives signals from the pressure sensor and the flow sensor, and when the measured mouth pressure exceeds a predetermined value, or when said pressure sensor or said flow sensor detects a pre-determined increase in pressure or flow, the control unit sends a signal to activate said pump or fan for creating an exhalation flow;
the flow is measured and controlled thus that said pump or fan for creating a flow, when activated, maintains a targeted flow at least at one pre-determined flow value, substantially independent of exhalation pressure; and
said pump or fan is deactivated when a predetermined time has lapsed, when a predetermined exhalation volume has been exhaled, or when the pressure sensor detects a pre-determined decrease in mouth pressure.

11. The method according to claim 10, wherein the pressure sensor activates said pump or fan for creating an exhalation flow when said pressure sensor detects a pre-determined increase in mouth pressure.

12. The method according to claim 10, wherein the exhalation flow rate is maintained at or around at least one value within the interval of about 1 to about 1000 ml/s.

13. The method according to claim 10, wherein the exhalation flow rate is maintained at or around at least one value within the interval of about 5 to about 600 ml/s, or about 5 to about 400 ml/s, with an accuracy of at least +/−10% of desired value.

14. The method according to claim 10, wherein mouth pressure is maintained at 3-40 mbar.

15. The method according to claim 10, wherein said flow is maintained at two or more sequential and pre-determined flow values, each for a predetermined period of time or volume.

16. The method according to claim 10, further including the presentation of an incentive for the mammal to initiate and maintain exhalation into the device.

17. The method according to claim 10, wherein said mammal is a human.

18. The method according to claim 10, wherein said mammal is a human subject chosen from the group including infants, small children, elderly, demented, mentally or physically disabled, and healthy.

19. A device or system for taking a sample of exhaled air from a mammal, comprising an inlet for receiving exhaled air from said mammal, a flow channel, a pressure sensor, a flow sensor, a control unit, and a pump or fan for creating an exhalation flow, wherein
said pressure sensor and/or flow sensor are adapted to supply signals to the control unit, and
said control unit is adapted to control the pump or fan in such manner, that said pump or fan is activated when said pressure and/or flow sensor detects a predetermined increase in mouth pressure and/or flow, and deactivated when said pressure sensor detects a predetermined decrease in mouth pressure.

20. The device or system according to claim 19, wherein said pump or fan for creating an exhalation flow is activated when said pressure sensor detects a predetermined increase in mouth pressure generated by the mammal.

21. The device or system according to claim 19, wherein said pump or fan is adapted to maintaining a predetermined exhalation flow within the interval of about 1 to about 1000 ml/s.

22. The device or system according to claim 19, said pump or fan is adapted to maintaining a predetermined exhalation flow at or around at least one value within the interval of about 5 to about 600 ml/s with an accuracy of +/−10%.

23. The device or system according to claim 19, adapted to eliminate the contribution of nasal air by securing velum closure by maintaining a mouth pressure of at least 3 mbar.

24. The device or system according to claim 19, further comprising means for storing a sample.

25. The device or system according to claim 19, further adapted for being connected to a second device for analyzing one or more components in a sample of exhaled air.

26. The device or system according to claim 19, adapted for being connected upstream of said second device, i.e. between an exhaling mammal and said second device.

27. The device or system according to claim 19, adapted for being connected downstream of said second device.

28. A device or system for determining the concentration of a component in exhaled air from a mammal, comprising an inlet for receiving exhaled air from said mammal, a flow channel, a pressure sensor, a flow sensor, a control unit, a pump or fan for creating an exhalation flow and a sensor specific for the component to be determined, wherein
said pressure sensor and/or flow sensor are adapted to supply signals to the control unit, and
said control unit is adapted to control the pump or fan in such manner, that said pump or fan is activated when said pressure and/or flow sensor detects a predetermined increase in mouth pressure and/or flow, and deactivated when said pressure sensor detects a predetermined decrease in mouth pressure.

29. The device or system according to claim 28, wherein said pump or fan for creating an exhalation flow is connected to said pressure sensor in such manner, that said pump or fan is activated when said pressure sensor detects a predetermined increase in mouth pressure.

30. The device or system according to claim 28, wherein said means for creating an exhalation flow is a pump or fan adapted to maintaining a predetermined flow at or around at least one value within the interval of about 1 to about 1000 ml/s.

31. The device or system according to claim 28, adapted to maintain at least one predetermined flow with an accuracy of +/−10%.

32. The device or system according to claim 28, adapted to eliminate the contribution of nasal air by securing velum closure by maintaining a mouth pressure of at least 3 mbar.

33. The device or system according to claim 28, wherein said sensor specific for the component to be determined is a nitric oxide sensor chosen from colorimetric, ultrasonic, chemoluminescence and electrochemical nitric oxide sensors.

34. A method for the investigation of disorders of the nasal airways, wherein a sample is aspirated from the nasal airways at a flow rate of about 1-100 ml/s, or about 20-350 ml/s, or about 40-400 ml/s, or about 40-600 ml/s, and different sub-intervals thereof, while the velum or soft palate is kept closed.

35. The method according to claim 34, wherein the subject is instructed to take a deep breath and to hold their breath during the sampling.

36. The method according to claim 34, wherein the subject exhales against a resistance creating a mouth pressure sufficient to close the velum or soft palate during the sampling.

37. The method according to claim 34, wherein a low-resistance filter for removal of the marker to be determined is connected to one nostril while a sample is aspired from the other.

38. The method according to claim 34, wherein the marker is endogenous nitric oxide, and the disorder to be investigated is primary ciliary dyskinesia.

39. The method according to claim 34, using a system comprising a flow channel, a pressure sensor, a flow sensor, a control unit, a pump or fan for creating a flow, and a tube with a nasal adapter.

40. The method according to claim 34, wherein an increased level of endogenous nitric oxide in the nasal airways, as compared to healthy controls, is taken as an indication of allergic rhinitis.

41. The method according to claim 34, wherein a reduced level of endogenous nitric oxide in the nasal airways, as compared to healthy controls, is taken as an indication of primary ciliary dyskinesia or cystic fibrosis.

42. A device or system for aspirating a sample from the nasal airways comprising an inlet adapted to be inserted in a nostril, a flow sensor, a control unit, a pump or fan for creating an exhalation flow, wherein
said flow sensor is adapted to supply signals to the control unit, and
said control unit is adapted to control the pump or fan in such manner, that a sample is aspirated from the nasal airways at or around at least one flow rate in the interval of about 1-100 ml/s.

43. A device or system according to claim 42, for use in determining the presence and/or concentration of a marker substance in a sample from the nasal airways, wherein the device or system comprises a low-resistance filter for removal of the marker to be determined, connected to an inlet adapted to be inserted in a nostril, contralateral to the nostril from which a sample is aspirated.

44. A device or system according to claim 42, comprising means for maintaining a mouth pressure of at least 3 mbar (0.3 kPa) securing velum closure.
